(19) United States
(12) Patent Application Publication
(10) Pub. No.: US 2024/0219366 A1
(43) Pub. Date: Jul. 4, 2024

(54) A METHOD OF DETECTING ONE OR MORE MARKERS IN A PETROLEUM FUEL USING A PHOTOACOUSTIC DETECTOR

(71) Applicant: BASF SE, Ludwigshafen am Rhein (DE)

(72) Inventors: Hans REICHERT, Basel (CH); Oliver SEEGER, Ludwigshafen am Rhein (DE); Korinna DORMANN, Ludwigshafen am Rhein (DE)

(21) Appl. No.: 18/287,476

(22) PCT Filed: Apr. 13, 2022
(86) PCT No.: PCT/EP2022/059839

§ 371 Date: Oct. 19, 2023

(30) Foreign Application Priority Data
Apr. 20, 2021 (EP) 21169508.5

(51) Int. Cl. G01N 33/28; G01N 21/17; G01N 21/64

(52) U.S. Cl. CPC G01N 33/2882; G01N 21/1702; G01N 21/64

(57) ABSTRACT

The present invention relates to a method of detecting a counterfeit, or adulterated petroleum fuel, comprising: a) emitting a modulated light beam from a modulated light source to a marked petroleum fuel in a chamber, wherein the marked petroleum fuel comprising a fuel additive, a mixture of a fluid petroleum fuel and a marker, wherein the marker is selected from the group consisting of organic IR absorbing compounds and mixtures thereof; b) producing an acoustic signal from the marker in the chamber, in response to the emitted modulated light beam; c) detecting the acoustic signal via a sensor disposed in the chamber; d) transmitting the acoustic signal from the sensor to a processor based module; and determining the marker and a concentration of the marker in the marked petroleum fuel via the processor based module, from the acoustic signal.

The present invention relates to a method of detecting a counterfeit, or adulterated petroleum fuel, comprising:
a) emitting a modulated light beam from a modulated light source to a marked petroleum fuel in a chamber, wherein the marked petroleum fuel comprising a fuel additive, a mixture of a fluid petroleum fuel and a marker, wherein the marker is selected from the group consisting of organic IR absorbing compounds and mixtures thereof;
b) producing an acoustic signal from the marker in the chamber, in response to the emitted modulated light beam;
c) detecting the acoustic signal via a sensor disposed in the chamber;
d) transmitting the acoustic signal from the sensor to a processor based module; and
determining the marker and a concentration of the marker in the marked petroleum fuel via the processor based module, from the acoustic signal.

WO2008/115521 relates to a method of measuring fluorophore excited state lifetimes comprising initiating an excitation laser pulse at a dye to excite dye molecules of the dye from a ground state to an excited state and initiating a probing pulse at the dye molecules thereby generating a first set of photoacoustic waves at a first time delay resulting in a first intensity point.

US20100223980 relates to a rapid recirculation based integrity testing of porous material and to an apparatus and system for performing the same.

US20190110691 relates to a photoacoustic targeting system, comprising
a light source configured to emit pulsed light;
a micropipette electrode configured to deliver the pulsed light to a target cell;
an acoustic transducer configured to receive photoacoustic signals generated due to optical absorption of light energy by the target cell; and
a controller configured to determine a position of the micropipette electrode relative to the target cell based on the photoacoustic signals.

US2013060122 relates to a device and method of using the device to detect the presence and composition of clots and other target objects in a circulatory vessel of a living subject.

Fuel samples are generally required to be labelled for a variety of purposes such as to distinguish between taxed and untaxed fuel oils or as brand identification for organic based liquids. Conventionally, fuels have been differentiated by means of colour, for example by including an appropriate dyestuff in the fuel, since colour is the simplest way of identifying fuel either by eye or quantitatively using a spectrophotometer.

The fuel is ideally marked with trace amounts of material (<50 ppm) such that the properties conferred by the marker chemical do not affect the bulk liquid. It should also be possible to detect concentrations as low as 5% of the marker in fuel in cases were the fuel has been diluted with unmarked fuel.

A variety of compounds have been described for marking fuels in the aforementioned manner. Often the fuel is marked with a chemical which is initially colourless but which becomes coloured upon the addition of a “developer” compound. U.S. Pat. No. 5,498,808, WO96/02613, WO95/07460, EP0438734, WO95/00606, U.S. Pat. No. 5,205,840 and WO95/10581.

The variable nature of fuel products renders them a challenging medium for analysis. Fuels, depending on fuel type and production conditions, exhibit varying ratios of aromatic and aliphatic components as well as ethanol which is especially true for the increasing amount of biofuel grades. Moreover, the constituents present in fuel tend to change as the result of chemical reactions that occur overtime. Similarly, variability in fuel compositions arise from the addition of oxygenates (e.g., ethanol, MTBE, and the like) or biologically derived components such as biodiesel.

Changes in absorbance and emission bands can result from fluctuations in the structure of the solvation shell around a marker. Moreover, spectral shifts (both bathochromic and hypsochromic) in the absorption and emission bands are often induced by a change in solvent mixture or composition.

Similarly, the fluorescence quantum yield (the ratio of the number of photons emitted to the number of photons absorbed by the fluorophore) is dependent on the solvent in which the analysis is conducted. A variety of non-radiative relaxation pathways are available and impact the fluorescence efficiency through mechanisms of dynamic or static quenching. Additionally, the temperature of a sample at the time of measurement has an impact on the fluorescence intensity observed for a given quantity of a fluorophore in solution. Generally, an increase in temperature results in a decrease in the fluorescence quantum yield because of an increase in the non-radiative processes related to collisions with solvent molecules, intramolecular vibrations, and rotations.

An additional problem presented when analyzing for a marker in fuels is that of a native variable background. Fuels, based on production conditions, chemical composition of starting crude oil, added additives and age of the fuels at the time of analysis, exhibit a natural background comprising absorbance and emission, in particular in the UV and VIS range of the electromagnetic spectrum. This background is highly variable and further complicates the quantification of a taggant (marker).

Another problem encountered is the presence of colorants often added to fuels. It is fairly common throughout the world to add colorants to fuels; this practice is often employed to allow specific grades or brands of fuel to be visually identified by consumers. The absorption or emission of these dyes can impinge in the spectral response range of a marker, further complicating identification/quantification.

These effects and compositional differences have a dramatic impact on the ability to accurately quantify the amount of a marker present in a fuel of unknown pedigree.

### SUMMARY

The present invention relates to devices and methods for determining the presence and quantity of a marker in a liquid sample.

Aspects and embodiments of the invention are set forth in the claims.

The claimed method bears a double benefit for the evaluation in fuel marking:

The benefit of the photoacoustic measurement lays in the separate and independent mode of action. As the excitation results from an optical interaction, but the detection is done as acoustic measurement, these physical phenomena are orthogonal to each other and do not overlap. This lack of influence from optical side effects and noises, which could lead either to a strong background measurement, or even worse to a wrong concentration information are thus circumvented by the system itself.

As the marker(s) show a strong NIR absorbing effect, the components and their concentration can be read out in an optical way additionally to the photoacoustic measurement, thus enhancing the safety and accuracy of the method.

In one embodiment of the present invention the method of detecting a counterfeit or adulterated petroleum fuel, comprises
a) emitting a modulated light beam from a modulated light source to a marked petroleum fuel in a chamber, wherein the marked petroleum fuel comprising a fuel additive, a mixture of a fluid petroleum fuel and a marker, wherein the marker is selected from the group consisting of organic IR absorbing compounds and mixtures thereof;
b) producing an acoustic signal from the marker in the chamber, in response to the emitted modulated light beam;
c) detecting the acoustic signal via a sensor disposed in the chamber;
d) transmitting the acoustic signal from the sensor to a processor based module; and
determining the marker and a concentration of the marker in the marked petroleum fuel via the processor based module, from the acoustic signal.

Preferably, the marker is present in an amount of from about 0.1 ppb to about 100 ppm.

In step a) a photoacoustic chemical detector may be used, comprising a light source for emitting light comprising two or more discrete optical modes; a photoacoustic sensor optically coupled to the light source for receiving light emitted from the light source, and being configured to output a sensor signal in response to acoustic energy created when received light from the light source interacts with the portion of the petroleum fuel within the photoacoustic sensor; and a controller electrically coupled to the light source and the photoacoustic sensor, wherein a drive signal is supplied to the light source such that the light source controllably emits light comprising a plurality of discrete modes, where each mode has a defined frequency and intensity; the sensor signal output is read from the photoacoustic sensor; and the marker is detected in the portion of the petroleum fuel using the sensor signal.

The identifying step b) further may comprise comparing the determined concentration with a target concentration of the marker.

In another embodiment of the present invention the method of detecting a counterfeit or adulterated petroleum fuel comprises
a) photoacoustically analyzing a portion of the petroleum fuel for the presence of a marker, wherein the marker consists of a single organic IR absorbing compound, or a mixture of organic IR absorbing compounds; and
b) identifying the petroleum fuel as counterfeit, adulterated or authentic as a function of the determined concentration of the marker, wherein the petroleum fuel comprises a fuel additive, wherein the organic IR absorbing compound is present in an amount of from about 0.1 ppb to about 10,000 ppb.

The term “petroleum fuel” refers to products having a predominantly hydrocarbon composition, although they may contain minor amounts of oxygen, nitrogen, sulfur or phosphorus. As used herein, the term “petroleum fuel” includes crude oils, as well as products derived from petroleum refining processes. The petroleum fuel is preferably selected from the group consisting of gasoline, diesel fuel, biodiesel fuel, kerosene, heating oil, heavy fuel oil, liquefied petroleum gas, ethanol, and any combination thereof. More preferably, the petroleum fuel is selected from the group consisting of gasoline, diesel fuel, kerosene, and jet fuel, and even more preferably from the group consisting of gasoline and diesel fuel.

A suitable photoacoustic measuring system is, for example, described in US20150059434A1, EP1195597A1 and WO2014/132046A2.

The photoacoustic measuring system comprises
a chamber having a marked petroleum fuel comprising a fuel additive, a mixture of a fluid petroleum fuel and a marker, wherein the marker is selected from the group consisting of organic IR absorbing compounds and mixtures thereof;
a modulated light source for emitting a modulated light beam to the marked petroleum fuel to generate an acoustic signal due to the presence of the marker;
a sensor disposed proximate the chamber, for detecting the acoustic signal; and
a processor based module communicatively coupled to the sensor and configured to receive the acoustic signal from the sensor and determine the marker and a concentration of the marker in the marked petroleum fuel based on the acoustic signal.

The modulated light source comprises a laser source and a modulator device.

The modulator device receives a light beam from the laser source, modulates the light beam, and generates the modulated light beam having a first beam wavelength and a second beam wavelength.

Preferably, the modulator device modulates at least one of an amplitude, frequency, and phase of the light beam.

The first beam wavelength and the second beam wavelength may be generated alternately.

The pressure sensor is at least one of a piezo effect based sensor, a cantilever based sensor, a microphone, a hydrophone, a capacitance based sensor, and a membrane based sensor.

The modulated light source may comprise a light source, at least one filter, and a modulator device for controlling at least one of an intensity of a light beam generated from the light source, a wavelength of the light beam, and a parameter of the light source.

Alternatively, the photoacoustic chemical detector may comprise a light source for emitting light comprising two or more discrete optical modes; a photoacoustic sensor optically coupled to the light source for receiving light emitted from the light source, and being configured to output a sensor signal in response to acoustic energy created when received light from the light source interacts with a sample of the petroleum fuel contained within the photoacoustic sensor, the sample of the petroleum fuel comprises a marker, wherein the marker consists of a single organic IR absorbing compound, or a mixture of organic IR absorbing compounds.

A method of detecting a counterfeit or adulterated petroleum fuel
using a the above-described photoacoustic chemical detector, comprises:
supplying a drive signal to the light source such that the light source controllably emits light comprising a plurality of discrete modes, where each mode has a defined frequency and intensity;
reading the sensor signal output from the photoacoustic sensor; and detecting one or more IR absorbing compounds in the sample of the petroleum fuel using the sensor signal.

The organic IR absorbing compounds may have sufficiently strong absorption and/or fluorescence in the near infrared (see, for example WO201250844, or U.S. Pat. No. 5,998,211), so that detection of the absorption by means of conventional photometers which are sensitive in this range and/or of the fluorescence by means of conventional instruments after excitation with a suitable radiation source is possible (spectroscopical analysis).

The method may comprise spectroscopically analyzing a portion of the petroleum fuel for the presence of a marker, wherein the marker consists of a single organic IR absorbing compound, or a mixture of organic IR absorbing compounds; determining a concentration of the organic IR absorbing compound(s) present in the portion of the petroleum fuel; comparing the determined concentration with a target concentration; and identifying the petroleum fuel as counterfeit, diluted, or authentic as a function of the determined concentration of the organic IR absorbing compound(s). The organic IR absorbing compound(s) are preferably present at a level of between 0.1 and 10,000 ppb.

In principle any IR absorbing organic compound known in the art, which has a main absorption maximum in the range from 700 to 1100 nm is suitable to be used as marker.

Preference is given to IR absorbing compounds which are “colourless”, which means that they have a minimal absorption in the VIS range of the electromagnetic spectrum, in particular in the range from 400 to 700 nm.

NIR absorbing compounds in terms of the present invention are polyunsaturated polycyclic organic compounds or metal organic compounds, which have a main absorption maximum in the range from 700 to 1100 nm. Particular preference is given to polycyclic organic compounds, in particular to complexes of mono- or polyunsaturated mono- or polycyclic organic compounds. NIR absorbing compounds are preferably metal-free and soluble in the application medium.

Examples of NIR compounds are squaric and croconic acid derivatives, quinone imides, especially (metal-free) phthalocyanines, (metal-free) naphthalocyanines, anthraquinone based dyes, boron azadipyrromethene dyes (see, for example, EP2480639), boron dipyrromethene dyes, azulenesquaric acid dyes, such as, for example, compounds of formula

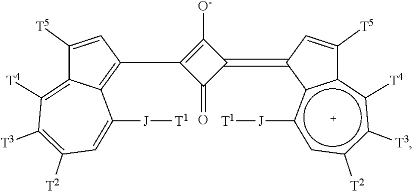

which are described in more detail in U.S. Pat. No. 5,998,211, polymethine dyes, such as, for example compounds of formula

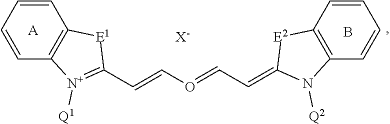

which are described in more detail in U.S. Pat. No. 5,998,211, rylene derivatives, such as, for example compounds of formula

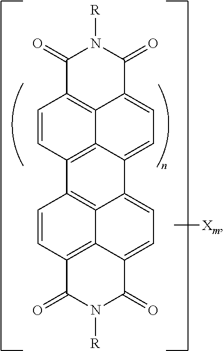

which are described in more detail in US20100011656A1, violanthrones, such as, for example dibenzanthrone and isodibenzanthrone derivatives, which are described in more detail in US20080194446; pyrrolopyrrols, such as, for example compounds of formula

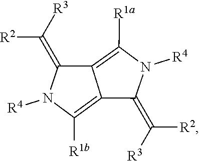

which are described in more detail in EP2272849, or mixtures thereof.

In a preferred embodiment of the present invention the organic IR absorbing compound(s) contained in the marked petroleum fuel of the present invention is selected from the group consisting of dibenzanthrone derivatives of the formula

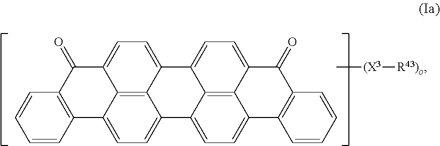

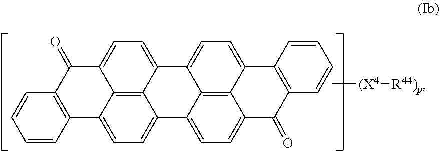

wherein X³, X⁴ are each independently —O—, —S—, —NH—, —NY¹—, —CO—, —O—CO—, —CO—O—, —S—CO—, —CO—S—, —NH—CO—, —CO—NH—, —NY¹—CO—, —CO—NY¹—, —CH₂—NH—, —CH₂—NY¹—, —CH₂—NH—CO— or —CH₂—NY¹—CO—, where the latter four groups mentioned are each bonded via the CH₂ group to the basic dibenzanthrone, or isodibenzanthrone structure,
R⁴³, R⁴⁴, Y¹ are each independently C₁-C₂₀alkyl which is optionally interrupted by from 1 to 4 oxygen atoms in ether function; C₅-C₇cycloalkyl which is optionally substituted by one or more C₁-C₂₀-alkyl groups which are optionally interrupted by from 1 to 4 oxygen atoms in ether function; saturated heterocyclic five- or six-membered radical which is optionally substituted by one or more C₁-C₂₀-alkyl groups which are optionally interrupted by from 1 to 4 oxygen atoms in ether function; C₆-C₁₀aryl which is optionally substituted by one or more halogen, cyano, nitro, hydroxyl, amino, C₁-C₂₀alkyl which is optionally interrupted by from 1 to 4 oxygen atoms in ether function, C₁-C₂₀-alkoxy, C₁-C₂₀-alkylamino or di(C₁-C₂₀-alkyl)amino; heteroaryl which has from 3 to 12 carbon atoms and may optionally be substituted by one or more C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino; C₆-C₁₀-aryl-C₁-C₄-alkyl which is optionally substituted in the aryl radical by one or more halogen, cyano, nitro, hydroxyl, amino, C₁-C₂₀-alkyl which is optionally interrupted by from 1 to 4 oxygen atoms in ether function, C₁-C₂₀alkoxy, C₁-C₂₀alkylamino or di(C₁-C₂₀alkyl)amino; or heteroarylC₁-C₄-alkyl having from 3 to 12 carbon atoms in the heteroaryl radical, the latter optionally being substituted by one or more C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino, and
o, p are integers from 1 to 16, where, when o>1 or p>1, the o (X³—R⁴³) moieties or the m (X⁴—R⁴⁴) moieties may be the same or different;
naphthalocyanine complexes of the formula

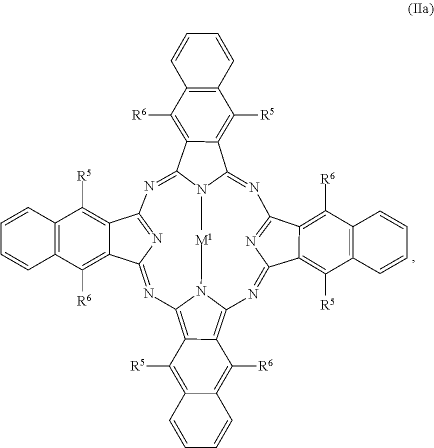

wherein
M¹ is two hydrogen atoms,
R⁵ is OR⁹, SR⁹, NHR¹⁰, or NR¹⁰R^{10′},
R⁶ is OR⁹, SR⁹, NHR¹⁰, or NR¹⁰R^{10′},
R⁹ is selected from the group consisting of C₁-C₁₂-alkyl, (C₂H₄O)ₘ₁—R^{10″} and phenyl;
R¹⁰, R^{10′} independently of each other are selected from the group consisting of C₁-C₁₂-alkyl, (C₂H₄O)ₙ₁—R^{10″} and phenyl, or
R¹⁰, R^{10′} together form a 5- or 6-membered saturated N-heterocyclic ring, which is optionally substituted by 1 or 2 methyl groups;
R^{10″} is C₁-C₁₂-alkyl, and
n1, m1 independently of each other are 0, 1, 2, 3 or 4;
phthalocyanine complexes of the formula

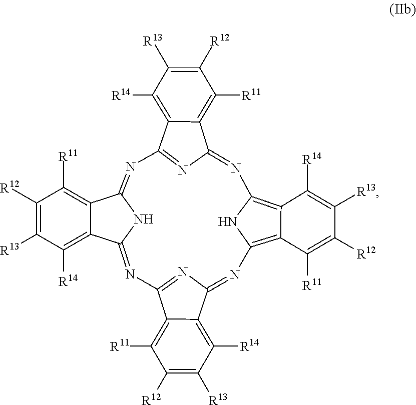

wherein
R¹¹ and R¹⁴ are independently of each other H, F, OR¹⁶, SR¹⁶, or NR¹⁷R^{17′},
R¹² and R¹³ are independently of each other H, F, OR¹⁶, SR¹⁶, NHR¹⁷, or NR¹⁷R^{17′},
R¹⁶ is C₁-C₁₂alkyl, (C₂H₄O)_{n′}OR¹⁸, or phenyl;
R¹⁷ and R^{17′} are independently of each other C₁-C₁₂alkyl, (C₂H₄O)_{n′}OR¹⁸, or phenyl; or
R¹⁷ and R^{17′} together may represent a 5- or 6-membered aliphatic ring, wherein one C-atom in the ring may be replaced by oxygen, to form a pyrrolidine, piperidine, 2-methylpiperidine or morpholine radical;
R¹⁸ is C₁-C₁₂alkyl;
n′ is 0 1, 2, 3 or 4;
compounds of formula

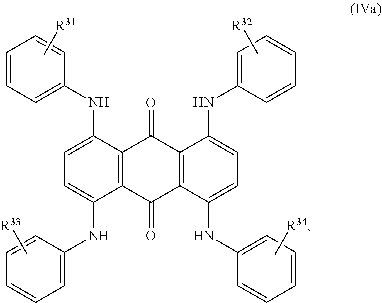

wherein
R³¹, R³², R³³ and R³⁴ are independently of each other C₁-C₆alkyl, or C₁-C₄alkoxy;
compounds of formula

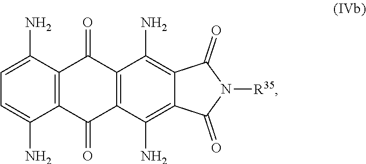

compounds of formula

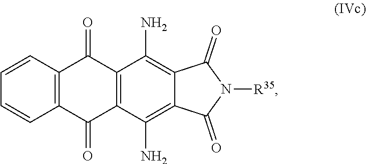

wherein R³⁵ is C₁-C₁₈alkyl, which can optionally be interrupted by 2 to 4 oxygen atoms;
compounds of formulae

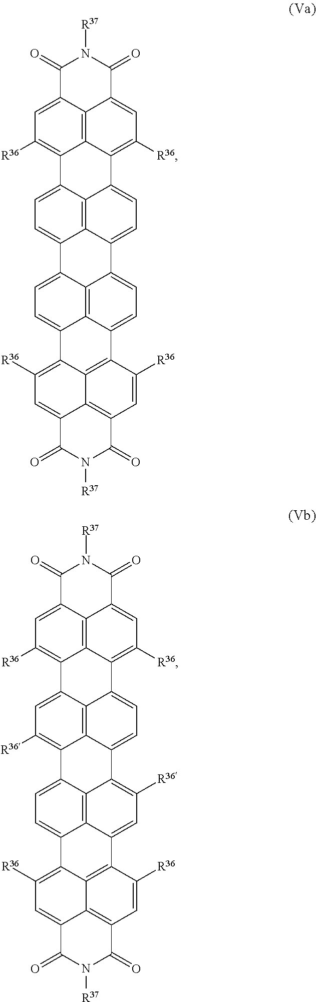

wherein
R³⁶ is H, X²R³⁸, or NR³⁸R³⁹;
R^{36′} is H, Br, X²R³⁸, or NR³⁸R³⁹;
X² is O, S, or NH;
R³⁸ is C₁-C₄alkyl or phenyl which phenyl can optionally be substituted by C₁-C₁₃alkyl;
R³⁹ is H, or C₁-C₄alkyl;
R³⁷ is C₁-C₁₃alkyl, phenyl, or 2,6-diisopropylphenyl;
compounds of formula

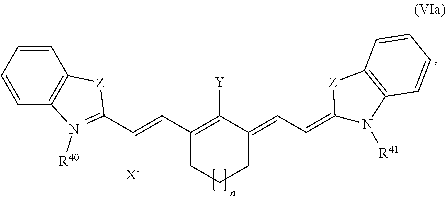

compounds of formula

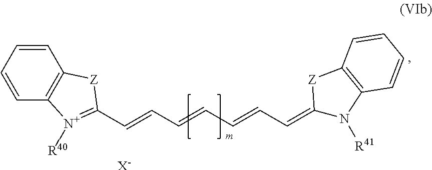

compounds of formula

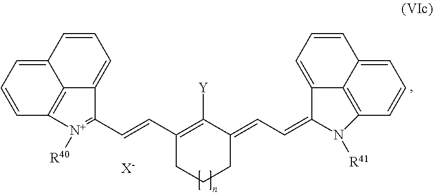

wherein
R⁴⁰ and R⁴¹ are independently of each other C₁-C₁₈alkyl;
Y is Cl, phenyl, 4-dimethylaminopyridyl chloride;
Z is O, S, NMe, or C(CH₃)₂,
n is 0, or 1;
m is 0, 1, or 2; and
X⁻=I⁻, BF₄⁻, PF₆⁻, R⁴²—C₆H₄—SO₃⁻, and
R⁴² is H, or CH₃;
compounds of formula

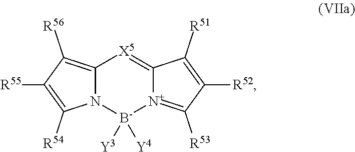

wherein
R⁵¹, R⁵², R⁵³, R⁵⁴, R⁵⁵ and R⁵⁶ are independently of each other hydrogen, or linear, or branched C₁-C₄alkyl groups, or R⁵² and R⁵⁵ are CN, or the R⁵¹ and R⁵² and the R⁵⁵ and R⁵⁶ pairs are part of a fused aromatic ring system,
X⁵ is N, or a group CR⁵⁷, wherein R⁵⁷ is a linear, or branched C₁-C₁₀alkyl group, and Y³ and Y⁴ are independently chosen from halogens, C₁-C₄alkyl groups, C₂-C₄alkenyl groups, or an optionally substituted phenyl group, especially F and mixtures thereof.

In a preferred embodiment of the present invention the organic IR absorbing compound is a compound of formula (IIa). If R⁵ and R⁶ have different meanings, formula (IIa) represents a simplified structure. While each group R⁵ formula (IIa) stands next to a group R⁶, R⁵ may be arranged next to a group R⁵ as well as R⁶ may be arranged next to a group R⁶.

The radicals R⁵ and R⁶ in formula (IIa), independently of one another, are preferably selected from the group consisting of OR⁹ and NR¹⁰R^{10′}, in particular from OR⁹.

Preferably, the radicals R⁵ and R⁶ have the same meaning.

The radicals R⁹, R¹⁰, R^{10′}, R^{10″}, n1 and m2 have the following preferred meanings:
R⁹ is C₁-C₈-alkyl or (C₂H₄O)ₘ₁—R^{10″}, in particular (C₂H₄O)ₘ₁—R^{10″};
R¹⁰ and R^{10′}, independently of each other, are C₁-C₈-alkyl or (C₂H₄O)ₙ₁—R^{10″}, more preferably C₁-C₆-alkyl or (C₂H₄O)ₙ₁—R^{10″} with n1 and R^{10″} having the preferred meanings defined herein, or R¹⁰ and R^{10′} together form a 5- or 6-membered saturated N-heterocyclic ring;
R^{10″} is C₁-C₈-alkyl, in particular C₁-C₆-alkyl;
n1 and m1, independently of each other, are 1, 2 or 3, in particular 2 or 3.

In another preferred embodiment of the present invention the organic IR absorbing compound is a compound of formula (IVa), in particular a compound of formula

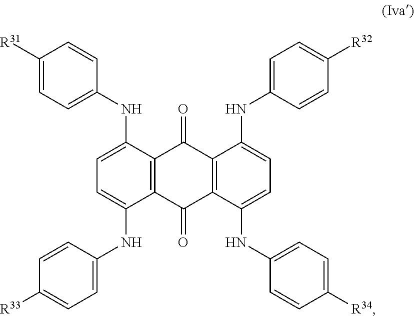

wherein R³¹, R³², R³³ and R³⁴ are independently of each other C₁-C₆alkyl and are preferably the same.

In another preferred embodiment of the present invention the organic IR absorbing compound is a compound of formula (Ia), or (Ib), in particular an isodibenzanthrone derivative of the formula

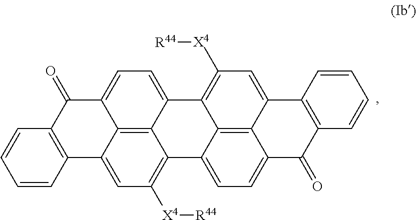

wherein X⁴ is —O—, and R⁴⁴ is a C₁-C₂₀alkyl group.

Examples of particular preferred organic IR absorbing compounds are cpd. A-1, cpd. A-2, cpd. A-3, cpd. A-4 (see Example 3 of U.S. Pat. No. 6,215,008), cpd. A-5 and cpd. A-6 shown in claim 10.

Various aspects and features of the present invention will be further discussed in terms of the examples. The following examples are intended to illustrate various aspects and features of the present invention.

### EXAMPLE 1—DETECTION OF KEROSENE IN DIESEL FUEL

Governments of countries often subsidize a fuel product such as kerosene to provide a low cost fuel for economically depressed households for a source of energy for cooking and lighting. However, these programs are often subject to widespread abuse. Subsidized kerosene is sold at much lower prices than gasoline or diesel and is frequently diverted by corrupt groups for use as a transport fuel.

For this example, a kerosene sample was dosed at 200 parts per billion (ppb) (w/w) with an organic IR absorbing compound,

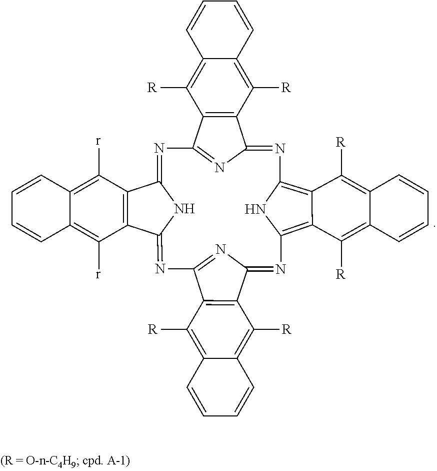

The marked kerosene was then added into samples of five different diesel fuels of varying origin. The diluted diesel sample was then analyzed.

Photoacoustic signals were measured using a photoacoustic spectroscopy (PAS) measurement system, as shown in FIG. 3 of M. J. Duffy et al., Photoacoustics 9 (2018) 49-61.

In addition, the above-mentioned compounds were detected marked liquids by absorption and by fluorescence, even if the compounds are only present in a concentration of approximately 0.1 ppm (detection by absorption) or approximately 5 ppb (detection by fluorescence).

Similarly, favorable results are achieved when naphthalocyanines of the above formula (where (R=O-n-C₈H₁₇; cpd. A-2, or R=O-n-C₁₂H₂₅; cpd. A-3).

1.-14. (canceled)

15. A system, comprising:
a chamber having a marked petroleum fuel comprising a fuel additive, a mixture of a fluid petroleum fuel and a marker, wherein the marker is selected from the group consisting of organic IR absorbing compounds and mixtures thereof;
a modulated light source for emitting a modulated light beam to the marked petroleum fuel to generate an acoustic signal due to the presence of the marker;
a sensor disposed proximate the chamber, for detecting the acoustic signal; and
a processor based module communicatively coupled to the sensor and configured to receive the acoustic signal from the sensor and determine the marker and a concentration of the marker in the marked petroleum fuel based on the acoustic signal.

16. A method of detecting a counterfeit or adulterated petroleum fuel, comprising:
a) emitting a modulated light beam from a modulated light source to a marked petroleum fuel in a chamber, wherein the marked petroleum fuel comprising a fuel additive, a mixture of a fluid petroleum fuel and a marker, wherein the marker is selected from the group consisting of organic IR absorbing compounds and mixtures thereof;
b) producing an acoustic signal from the marker in the chamber, in response to the emitted modulated light beam;
c) detecting the acoustic signal via a sensor disposed in the chamber;
d) transmitting the acoustic signal from the sensor to a processor based module; and
determining the marker and a concentration of the marker in the marked petroleum fuel via the processor based module, from the acoustic signal.

17. A method of detecting a counterfeit or adulterated petroleum fuel, the method comprising:
a) photoacoustically analyzing a portion of the petroleum fuel for the presence of a marker, wherein the marker consists of a single organic IR absorbing compound, or a mixture of organic IR absorbing compounds; and
b) identifying the petroleum fuel as counterfeit, adulterated or authentic as a function of the determined concentration of the marker, wherein the petroleum fuel comprises a fuel additive, wherein the organic IR absorbing compound is present in an amount of from about 0.1 ppb to about 10,000 ppb.

18. The system according to claim 15, wherein the marker is present in an amount of from about 0.1 ppb to about 100 ppm.

19. The system according to claim 15, wherein the petroleum fuel is selected from the group consisting of gasoline diesel fuel, biodiesel fuel, kerosene, liquefied petroleum gas, ethanol, and any combination thereof.

20. The system according to claim 15, wherein the organic IR absorbing compound is selected from the group consisting of squaric and croconic acid derivatives, quinone imides, especially (metal-free) phthalocyanines, (metal-free) naphthalocyanines, anthraquinone based dyes, boron azadipyrromethene dyes, boron dipyrromethene dyes, azulenesquaric acid dyes, polymethine dyes, rylene derivatives, violanthrones, such as, for example dibenzanthrone and isodibenzanthrone derivatives; pyrrolopyrrols, or mixtures thereof.

21. The system according to claim 15, wherein the organic IR absorbing compound is selected from the group consisting of dibenzanthrone derivatives of the formula 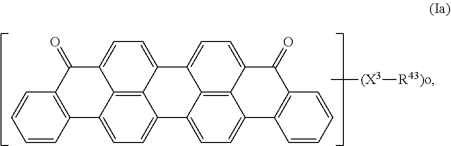
isodibenzanthrone derivatives of the formula 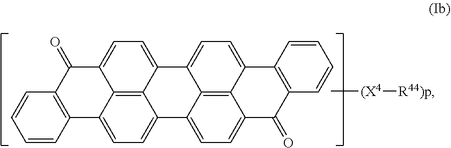
wherein X³, X⁴ are each independently —O—, —S—, —NH—, —NY¹—, —CO—, —O—CO—, —CO—O—, —S—CO—, —CO—S—, —NH—CO—, —CO—NH—, —NY¹—CO—, —CO—NY¹—, —CH₂NH—, —CH₂NY¹—, —CH₂NH—CO— or —CH₂—NY¹—CO—, where the latter four groups mentioned are each bonded via the CH₂ group to the basic dibenzanthrone, or isodibenzanthrone structure,
R⁴³, R⁴⁴, Y¹ are each independently C₁-C₂₀alkyl which is optionally interrupted by from 1 to 4 oxygen atoms in ether function; C₅-C₇cycloalkyl which is optionally substituted by one or more C₁-C₂₀-alkyl groups which are optionally interrupted by from 1 to 4 oxygen atoms in ether function; saturated heterocyclic five- or six-membered radical which is optionally substituted by one or more C₁-C₂₀-alkyl groups which are optionally interrupted by from 1 to 4 oxygen atoms in ether function; C₆-C₁₀aryl which is optionally substituted by one or more halogen, cyano, nitro, hydroxyl, amino, C₁-C₂₀alkyl which is optionally interrupted by from 1 to 4 oxygen atoms in ether function, C₁-C₂₀-alkoxy, C₁-C₂₀-alkylamino or di(C₁-C₂₀-alkyl)amino; heteroaryl which has from 3 to 12 carbon atoms and may optionally be substituted by one or more C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino; C₆-C₁₀-aryl-C₁-C₄-alkyl which is optionally substituted in the aryl radical by one or more halogen, cyano, nitro, hydroxyl, amino, C₁-C₂₀-alkyl which is optionally interrupted by from 1 to 4 oxygen atoms in ether function, C₁-C₂₀alkoxy, C₁-C₂₀alkylamino or di(C₁-C₂₀alkyl)amino; or
heteroarylC₁-C₄-alkyl having from 3 to 12 carbon atoms in the heteroaryl radical, the latter optionally being substituted by one or more C₁-C₂₀-alkyl which is optionally interrupted by from 1 to 4 oxygen atoms in ether function, C₁-C₂₀-alkoxy, C₁-C₂₀-alkylamino or di(C₁-C₂₀-alkyl)amino, and
o, p are integers from 1 to 16, where, when o>1 or p>1, the o (X³—R⁴³) moieties or the m (X⁴—R⁴⁴) moieties may be the same or different;
naphthalocyanine complexes of the formula 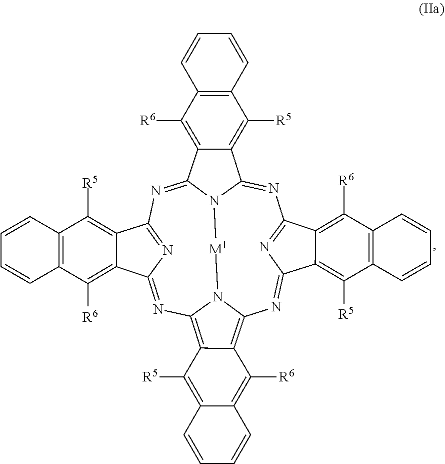
wherein
M¹ is two hydrogen atoms,
R⁵ is OR⁹, SR⁹, NHR¹⁰ or NR¹⁰R^{10′},
R⁶ is OR⁹, SR⁹, NHR¹⁰, or NR¹⁰R^{10′},
R⁹ is selected from the group consisting of C₁-C₁₂-alkyl, (C₂H₄O)ₘ₁—R^{10″} and phenyl;
R¹⁰, R^{10′} independently of each other are selected from the group consisting of C₁-C₁₂-alkyl, (C₂H₄O)ₙ₁—R^{10″} and phenyl, or
R¹⁰, R^{10′} together form a 5- or 6-membered saturated N-heterocyclic ring, which is optionally substituted by 1 or 2 methyl groups;
R^{10″} is C₁-C₁₂-alkyl, and
n1, m1 independently of each other are 0, 1, 2, 3 or 4;
phthalocyanine complexes of the formula 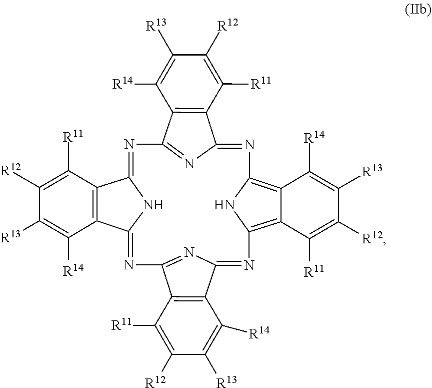
wherein
R¹¹ and R¹⁴ are independently of each other H, F, OR¹⁶, SR¹⁶, or NR¹⁷R^{17′},
R¹² and R¹³ are independently of each other H, F, OR¹⁶, SR¹⁶, NHR¹⁷ or NR¹⁷R^{17′},
R¹⁶ is C₁-C₁₂alkyl, (C₂H₄O)_{n′}OR¹⁸, or phenyl;
R¹⁷ and R^{17′} are independently of each other C₁-C₁₂alkyl, (C₂H₄O)_{n′}OR¹⁸, or phenyl; or
R¹⁷ and R^{17′} together may represent a 5- or 6-membered aliphatic ring, wherein one C-atom in the ring may be replaced by oxygen, to form a pyrrolidine, piperidine, 2-methylpiperidine or morpholine radical;
R¹⁸ is C₁-C₁₂alkyl;
n′ is 0 1, 2, 3 or 4;
compounds of formula 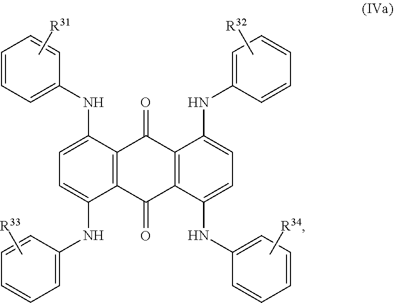
wherein
R³¹, R³², R³³ and R³⁴ are independently of each other C₁-C₆alkyl, or C₁-C₄alkoxy; compounds of formula 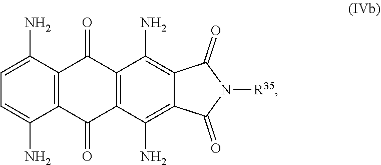
compounds of formula 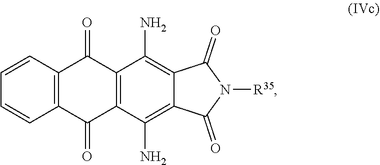
wherein R³⁵ is C₁-C₁₈alkyl, which can optionally be interrupted by 2 to 4 oxygen atoms;
compounds of formulae 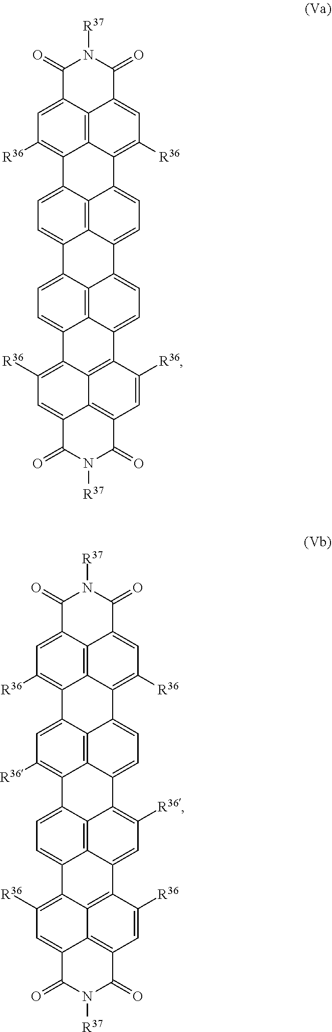
wherein R³⁶ is H, X²R³⁸, or NR³⁸R³⁹;
R^{36′} is H, Br, X²R³⁸, or NR³⁸R³⁹,
X² is O, S, or NH;
R³⁸ is C₁-C₄alkyl or phenyl which phenyl can optionally be substituted by C₁-C₁₈alkyl;
R³⁹ is H, or C₁-C₄alkyl;
R³⁷ is C₁-C₁₈alkyl, phenyl, or 2,6-diisopropylphenyl;
compounds of formula 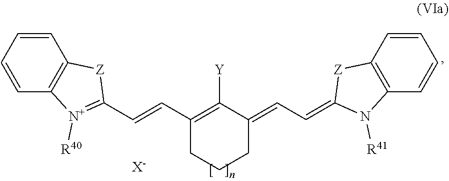
compounds of formula 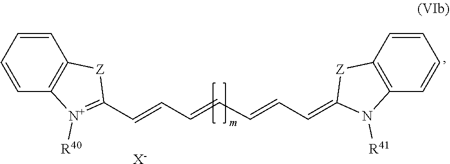
compounds of formula 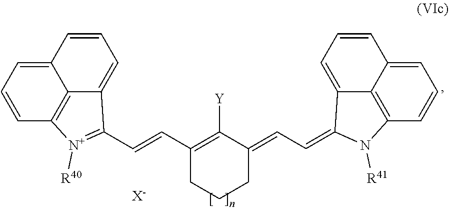
wherein
R⁴⁰ and R⁴¹ are independently of each other C₁-C₁₈alkyl;
Y is Cl, phenyl, 4-dimethylaminopyridyl chloride;
Z is O, S, NMe, or C(CH₃)₂,
n is 0, or 1;
m is 0, 1, or 2; and
X⁻=I⁻, BF₄⁻, PF₆⁻, R⁴²—C₆H₄—SO₃⁻, and
R⁴² is H, or CH₃;
compounds of formula 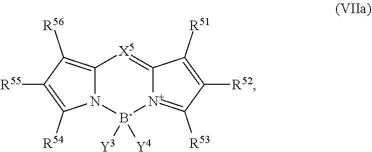
wherein
R⁵¹, R⁵², R⁵³, R⁵⁴, R⁵⁵ and R⁵⁶ are independently of each other hydrogen, or linear, or branched C₁-C₄alkyl groups, or the R⁵¹ and R⁵² and the R⁵⁵ and R⁵⁶ pairs are part of a fused aromatic ring system,
X⁵ is N, or a group CR⁵⁷, wherein R⁵⁷ is a linear, or branched C₁-C₁₀alkyl group, and Y³ and Y⁴ are independently chosen from halogens, C₁-C₄alkyl groups, C₂-C₄alkenyl groups, or an optionally substituted phenyl group, especially F and mixtures thereof.

22. The system according claim 21, wherein the organic IR absorbing compound is selected from
isodibenzanthrone derivatives of the formula 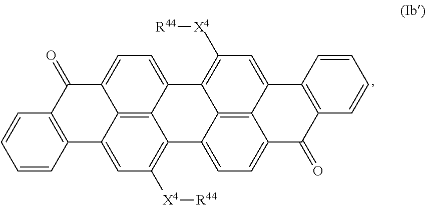
wherein X⁴ is —O—, and
R⁴⁴ is a C₁-C₂₀alkyl group;
naphthalocyanine complexes of the formula 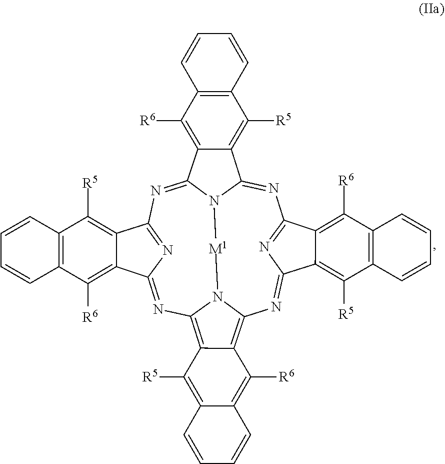
wherein
M¹ is two hydrogen atoms,
R⁵ is OR⁹,
R⁶ is OR⁹,
R⁹ is selected from the group consisting of a C₁-C₁₂alkyl group;
compounds of formula 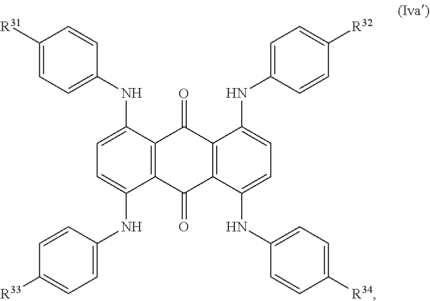
wherein R³¹, R³², R³³ and R³⁴ are independently of each other C₁-C₆alkyl.

23. The system according to claim 22, wherein the organic IR absorbing compound is selected from 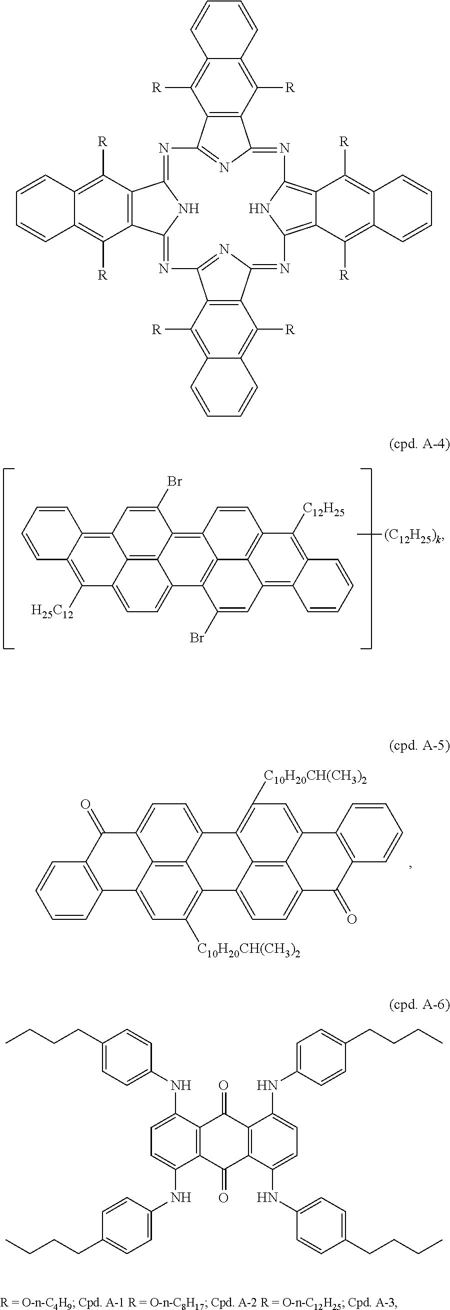
and mixtures thereof.

24. The system according to claim 15, wherein the marker is present in an amount of from about 500 ppb to about 10,000 ppb.

25. The system according to claim 15, wherein the organic IR absorbing compound has a main absorption maximum in the range from 700 to 1100 nm.

26. The method of claim 17, wherein in step a) a photoacoustic chemical detector is used, comprising a light source for emitting light comprising two or more discrete optical modes; a photoacoustic sensor optically coupled to the light source for receiving light emitted from the light source, and being configured to output a sensor signal in response to acoustic energy created when received light from the light source interacts with the portion of the petroleum fuel within the photoacoustic sensor; and a controller electrically coupled to the light source and the photoacoustic sensor, wherein a drive signal is supplied to the light source such that the light source controllably emits light comprising a plurality of discrete modes, where each mode has a defined frequency and intensity; the sensor signal output is read from the photoacoustic sensor; and the marker is detected in the portion of the petroleum fuel using the sensor signal.

27. The method according to claim 17, wherein the identifying step b) further comprises comparing the determined concentration with a target concentration of the marker.

28. The method according to claim 16, wherein the organic IR absorbing compounds have sufficiently strong absorption and/or fluorescence in the near infrared, so that detection of the absorption by means of conventional photometers which are sensitive in this range and/or of the fluorescence by means of conventional instruments after excitation with a suitable radiation source is possible.
